(19) United States
(12) Patent Application Publication
(10) Pub. No.: US 2010/0036336 A1
(43) Pub. Date: Feb. 11, 2010

(54) APPLICATION OF A WAIST BELT FOR AN ABSORBENT ARTICLE

(75) Inventors: Jenny BOGDANOVA, Goteborg (SE); Maria GUSTIN BERGSTROM, Halso (SE); Emilia KOVACIC, Vastra Frolunda (SE)

Correspondence Address: DRINKER BIDDLE & REATH (DC), 1500 K STREET, N.W., SUITE 1100, WASHINGTON, DC, 20005-1209, US

(73) Assignee: SCA HYGIENE PRODUCTS AB, Goteborg (SE)

(21) Appl. No.: 12/186,890

(22) Filed: Aug. 6, 2008

(51) Int. Cl. A61F 13/15

(52) U.S. Cl. 604/361; 604/385.03; 604/387; 604/392

(57) ABSTRACT

A garment and method of producing an absorbent article includes an absorbent section. The absorbent section includes a front and rear portion. A waist belt is attached to the absorbent section. The waist belt includes first and second belt portions extending on each side of the rear portion of the absorbent section for securing to each other around a wearer of the garment. The front portion of the absorbent section includes a fastening device for securing the front portion of the absorbent section to the waist belt. To better aid the wearer or caregiver on how to put the product on the user, instructions are provided directly on the garment which describe how the garment should be applied.

### FIELD OF THE INVENTION

The present invention relates to application of a waist belt for an absorbent garment. More particularly, the present invention relates to providing instructions to a user on how to apply the waist belt.

### BACKGROUND OF THE INVENTION

Garments in the form of disposable diapers are generally known in which the diaper is provided with a fastening system including a pair of fastening tabs secured to both sides of one end region of the diaper. The fastening tabs are intended to engage receiving means located on the other end region of the diaper. Such a diaper is generally placed on the wearer when the wearer is lying down. While the above-described fastening arrangement is satisfactory when applying diapers to a young child or baby, problems can arise when trying to fit an incontinence diaper to an adult wearer. This is particularly true for wearers who wish to apply the diaper to themselves, or for caregivers who receive a significant benefit from applying the diaper when the wearer is in a standing position.

To address this issue, a belted garment has been developed, which allows a wearer or caregiver to apply the diaper in a standing position. An example of such a belted garment is described in U.S. Pat. No. 6,342,050, which is incorporated herein by reference. Typically, the garment has a belt attached integrally with the absorbent garment. The belt may have two belt portions extending on either side of the rear end of the absorbent section. The two belt portions are intended to be fastened around the waist of the wearer while the front end of the absorbent section hangs down between the legs of the wearer. Once the belt portions have been joined together, the wearer can reach between his or her legs to draw up the absorbent section between the legs and to attach the free end of the absorbent section to the belt portion.

Similarly, the garment can be made so that the belt is fastened to the front portion of the product and is secured around the back of the wearer. In this case, the absorption product will be hanging down in the front and will be secured to the belt in the back. This type of product is particularly useful for caregivers who care for patients that may have dementia or the like. It should be noted that this type of configuration is not ideal for wearers who wish to apply the garment themselves.

Where the problem of incontinence is involved, it will be appreciated that persons suffering from this problem are often old and have physical handicaps of various types. As a consequence, they may have more difficulty fastening the belt by themselves and often require the assistance of personnel for fitting the garments. However, it has been discovered that both caregivers and users have difficulty understanding how the product should be applied. With regard to belted garments, the most important part of application is that the belt is attached first. Although directions of application may be included with the garment packaging, the instructions are often ignored or thrown away. Therefore, the user becomes frustrated with the product.

Accordingly, there is a need in the art to provide quick and easy instructions to a user or caregiver on how to apply a belted garment for absorption.

### SUMMARY

According to a first aspect of the invention, a method of producing an absorbent article includes providing a garment including an absorbent section, the absorbent section having a first and second end, a waist belt attached to the absorbent section, the waist belt having first and second belt portions extending on each side of the first end of the absorbent section for securing to each other around a wearer of the garment, the second end of the absorbent section including a fastening device for securing the second end of the absorbent section to the waist belt; and affixing instructions of application of the garment onto the garment.

According to a second aspect of the invention, a garment includes an absorbent section, the absorbent section having first and second ends, a waist belt attached to the absorbent section, the waist belt having first and second belt portions extending on each side of the first end of the absorbent section for securing to each other around a wearer of the garment, the second end of the absorbent section including a fastening device for securing the second end of the absorbent section to the waist belt, and instructions provided on the garment for describing how the garment should be applied.

According to a third aspect of the present invention, an absorbent article includes an absorbent section, the absorbent section having a first and second end, a fastening device attached to the absorbent section for securing the first end of the absorbent section to the second end of the absorbent section, and instructions provided on the article for describing how the article should be applied.

### BRIEF DESCRIPTION OF THE DRAWINGS

FIG. 1 is a top plan view of a belted garment to be provided with instructions according to the present invention.

FIG. 2A is a first pictorial representation of an instruction to be provided on the belted garment according to the present invention.

FIG. 2B is a second pictorial representation of an instruction to be provided on the garment according to the present invention.

FIG. 2C is a third pictorial representation of an instruction to be provided on the garment according to the present invention.

FIG. 3 is a top plan view of an outside of a belted garment with instructions provided on the longitudinal extension of the product according to the invention.

FIG. 4 is an example of a strip of instructions and information to be applied to a belted garment according to the invention.

FIG. 5 is a top plan view of an outside of a belted garment with instructions provided on the transversal extension of the product according to the present invention.

FIG. 6 is a top plan view of a belted garment with instructions provided along the inner waist edge of the product according to the present invention.

### DETAILED DESCRIPTION OF THE PREFERRED EMBODIMENTS

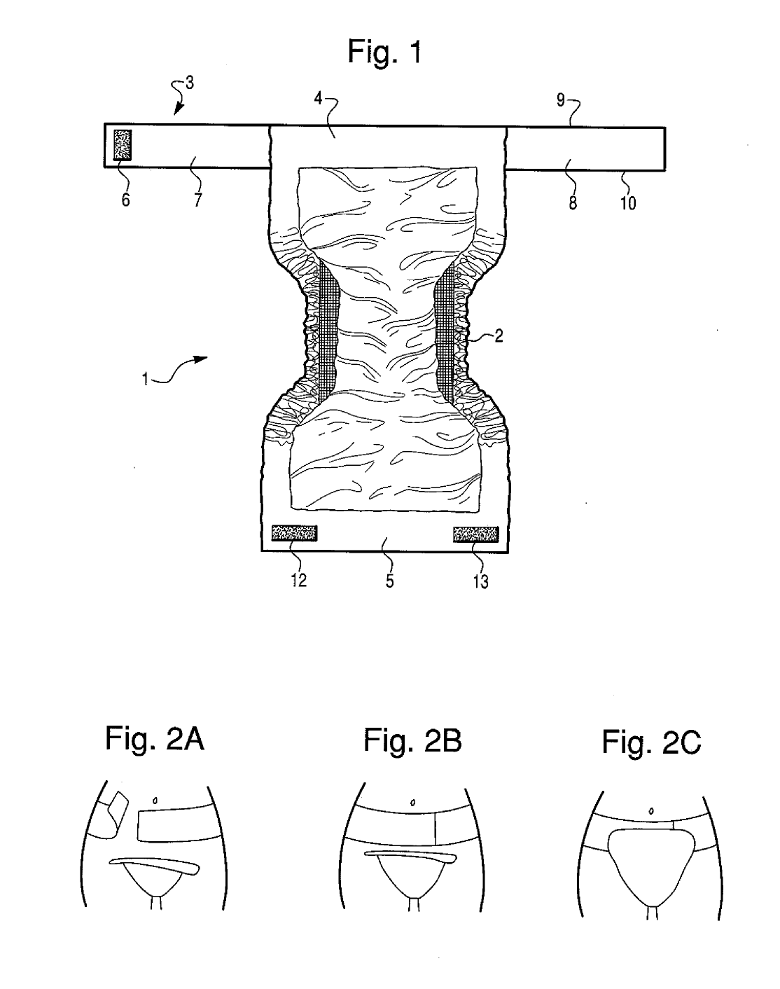

FIG. 1 shows a garment generally denoted 1 which includes an absorbent section 2 and a belt portion generally denoted 3. The belt could be either one continuous belt 3 attached to the absorbent section 2 at a rear end 4 or front end 5 thereof or could be two separate belt portions 7 and 8 each attached to a respective side of the rear end 4 or front end 5. The present invention pertains to providing instructions on how to use the garment so that the instructions are visible before use of the garment. In the preferred embodiment, the instructions are placed directly on the garment.

Before describing the instructions in detail, farther details of the belted garment 1 are provided according to features of the preferred embodiment. In particular, disposed at one end of belt portion 7 of the belt 3, there is provided a flexible strip 6 of hook elements, of the hook and loop type of fastening devices, which can either be secured to the belt part 8 or to a loop strip arranged on the belt part 8. However, it should be understood that other types of fastening devices are possible, including but not limited to, adhesive, pressure buttons, buttons and button holes, knots, and strings. In addition, while the belt 3 is preferably substantially rectangular in shape and includes two laterally spaced longitudinal edges 9 and 10, between which the strip 6 will be attached, other shapes are conceivable.

In the preferred embodiment, instructions relate to applying a belted garment having two separate belt portions 7 and 8. However, it should be understood that different instructions may be given, depending on the particular characteristics of the garment. In addition, it should be understood that the present invention could be used in connection with other incontinence guards or diapers.

With reference back to FIG. 1, disposed at a front end of the garment are two flexible strips 12 and 13 of hook elements, which can be secured directly to the belt 3. However, it should be understood that other types of fastening devices are possible, including but not limited to, adhesive, pressure buttons, buttons and button holes, knots, and strings. In use, the two belt portions 7 and 8 are intended to be fastened around the waist of the wearer while the front end 5 of the absorbent section 2 hangs down between the legs of the wearer. Once the belt portions 7 and 8 have been joined together, the wearer can reach between his or her legs to draw up the absorbent section 2 between the legs and to attach the free end 5 of the absorbent section 2 to the belt 3. However, the method of using the product is not intuitive, and therefore, instructions are needed. The particular instructions for using this product are often included in the packaging. However, both caregivers and adult users may not read the directions and often become frustrated when trying to apply the device. The present invention addresses these problems by providing a belted garment that includes directions provided on the garment 1 so as to be immediately visible to the user or caregiver. While the preferred embodiment describes a garment having a belt that is fastened to the back portion of the product so that a front end 5 is brought up between the legs of the user and attached to the belt 3, it should be understood that the product could also be made so that the belt is fastened to the front portion of the product and secured around the back of the wearer.

With reference to FIGS. 2A-2B, an example of instructions to be provided on the garment are described in more detail. In particular, pictorial instructions are preferably provided that easily instruct a user or caregiver on how to apply the product to a user. FIG. 2A shows a first position, wherein the belt 3 is open. As shown in the pictorial representation of FIG. 2A, the belt portion 8 is pulled around the waist of the user. The other belt portion 7 and absorbent section 2 remain free. With reference to FIG. 2B, the other belt portion 7 is pulled around the waist of the user, and then connected to the belt portion 8. The absorbent section 2 remains free. In the preferred embodiment, fastening the belt 3 around the user is the most important step of the process. Therefore, it is important that the instructions be readily apparent to the caregiver or user.

Finally, with reference to FIG. 2C, the absorbent section 2 is drawn upwards between the legs of the user and then fastened to the belt 3. Optionally, the pictorial representations shown in 2A-2C may also include a reference number or character to indicate the sequence in which the belt is to be fastened. For example, the pictorial representation of FIG. 2A may have a “1”, the pictorial representation of FIG. 2B may have a “2”, and the pictorial representation of FIG. 2C may have a “3”, to more clearly designate the sequence. Likewise, an “A”, “B” “C” sequence, or any other sequence, may also be used.

As described above, other types of instructions are possible, such as written instructions alone or written instructions together with pictorial instructions. In addition, the instructions may change depending on the particular structure and application of the garment. Accordingly, the present invention is not limited to the pictorial instructions described in the preferred embodiment. In addition, the instructions may be more or less detailed, or related to disposal of the product.

The instructions may be placed on the garment 1 in various places. Preferably, the instructions are placed near a waist edge of the product and should be visible before unfolding the product. However, it should be understood that instructions may be placed at any location on the garment or diaper, so long as it is relatively visible to the wearer or caregiver.

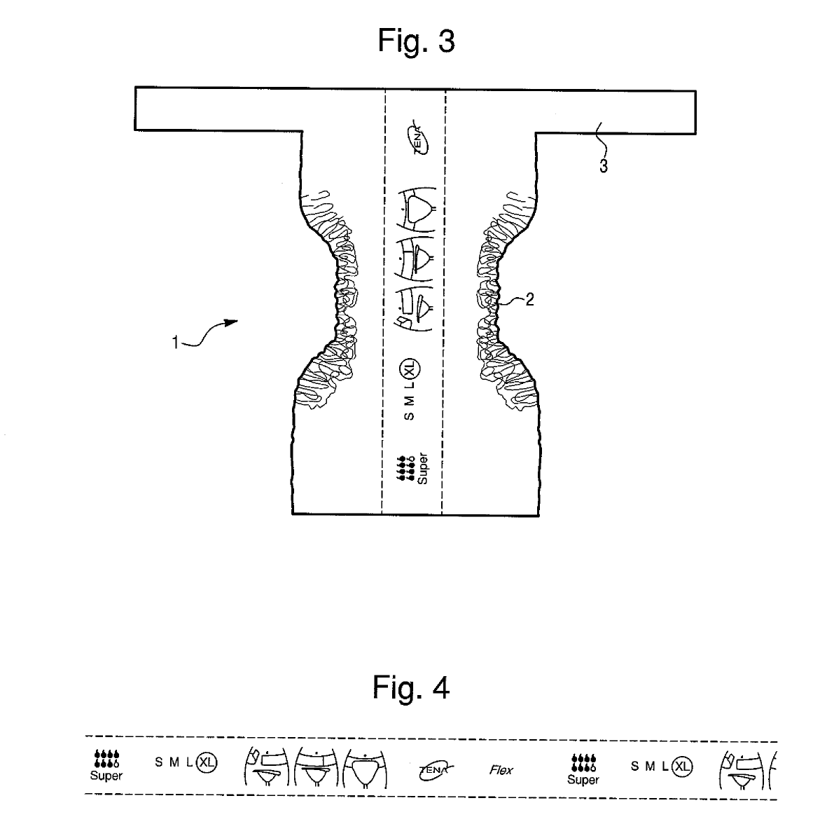

With reference to FIG. 3, the instructions 20 are provided along the longitudinal extension L of the garment 1. FIG. 4 shows an example of the instructions 20 together with the product name, size and absorption level. However, other types of information and design elements may also be provided. In addition, the instructions may be printed in one or more colors, as well as a different color from the information and design elements included with the instructions. However, the instructions, information and design elements may all be printed in the same color.

In the preferred embodiment, the information is repeated, as shown in FIG. 4. Most preferably, the instructions are repeated at least two times. However, it should also be understood that the instructions may be included just once or multiple times, by themselves, or together with additional information and design elements, depending on design preference. While the instructions 20 are shown in FIG. 3 as extending substantially along the longitudinal extension L of the product, it should be understood that the instructions may cover only a part of the longitudinal extension of the product,

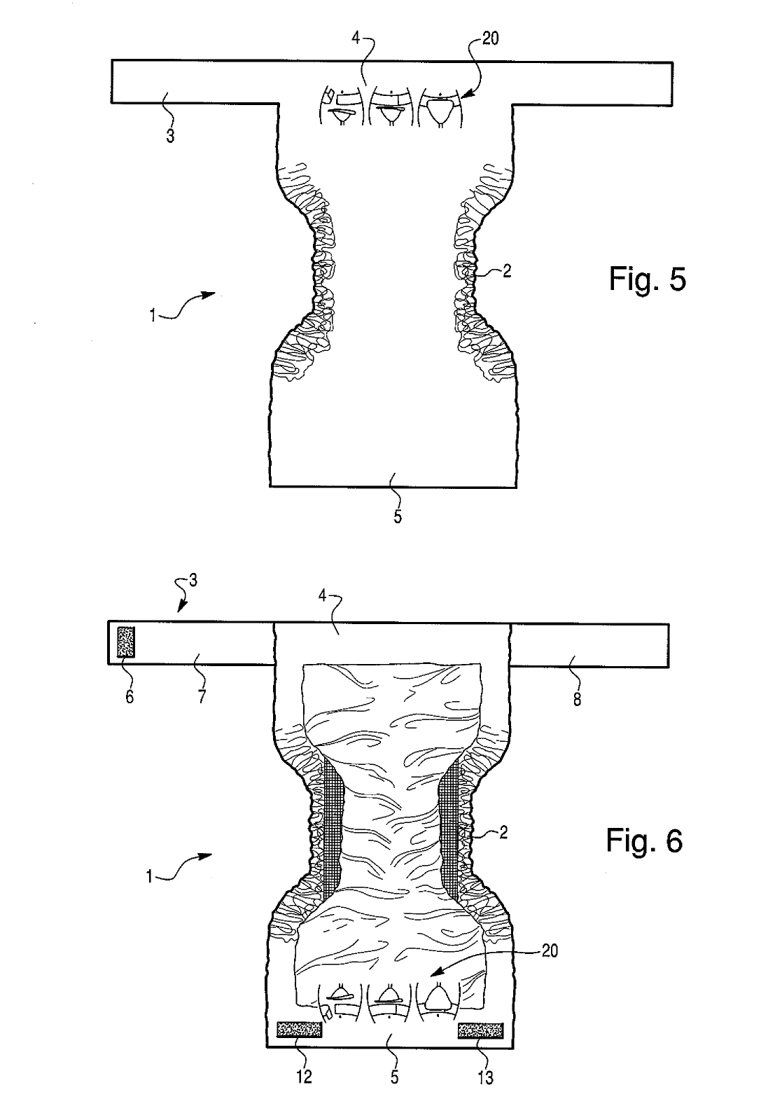

FIG. 5 shows an alternative embodiment of the present invention, showing placement of the instructions 20 on the outside of the product 1 along a rear end 4 of the absorbent section 2. According to this embodiment, the instructions extend along a transversal end T of the product. While the instructions shown in FIG. 5 extend substantially along the transversal extension T of the rear portion 4 of the product 1, it should be understood that the instructions may cover only a part of the transversal extension of the product, as well as along the entire front and rear end portions 5 and 4 or just along front portion 5. It should also be understood that the instructions 20 may be placed at multiple locations, as well as various directions, and at any location on the garment or diaper. The instructions may also be placed on the belt 3.

The instructions 20 may also be placed on the inside of the waist edge along the front portion 5 of the absorbent section 2, as shown, for example, in FIG. 6. In this embodiment, the instructions 20 are visible to the user once the product 1 is disposed between the legs of the user. The instructions may also be placed on the product strategically so that during the course of applying the garment or diaper, the instructions become visible at that particular point in the process.

The instructions may be placed on the product in various ways. In particular, the instructions 20 may be directly printed on the product 1, as shown for example with respect to FIG. 5, or they may be included on a separate sheet of material 22, and then affixed to the product 1, as shown for example with respect to FIG. 3. The separate sheet 22 may be permanently affixed, or removably affixed. The sheet 22 may be made from any kind of web-like material, as long as it is sufficiently soft. For example, the sheet 22 may be made from paper, but also nonwoven or plastic film and the like. See, also, U.S. Pat. No. 6,572,575. In the case of a removable sheet, this allows for more discretion for the user so that the instructions may be removed once the product is applied. Alternatively, the separate sheet of material may be placed in the product next to the backsheet so that it can be visible from the outside.

In addition, the actual print of the instructions may be made of a substance that changes state when wet, such as by dissolving, smearing or changing colors, thereby discreetly removing the print. As such, the print of the instructions could also act as a wetness indicator, which is well known in the art. For example, the instructions may be placed on the inside of the product. When the print dissolves, this will indicate that the absorbent product is wetted. Alternatively, the print may change, thereby indicating that the absorbent product is wetted. For example, the print of the instructions may dissolve and be replaced by an indicator advising that the product is wetted. Wetness indication systems described above are well known in the art as shown for example in U.S. Pat. No. 3,675,654 and U.S. Pat. No. 4,022,211, said patents of which are incorporated by reference herein.

It should be noted that, while the term “absorbent garment” has been used particularly in conjunction with incontinence, and particularly adult incontinence, the invention is not limited to this particular use or any particular size or type of absorbent garment implied thereby. The above-described product and method could be used with baby's or children's diapers.

Although the present invention has been described in connection with preferred embodiments thereof it will be appreciated by those skilled in the art that additions, deletions, modifications, and substitutions not specifically described may be made without department from the spirit and scope of the invention as defined in the appended claims.

1. A method of producing an absorbent article, comprising:
providing a garment including an absorbent section, the absorbent section having a first and second end, a waist belt attached to the absorbent section, the waist belt having first and second belt portions extending on each side of the first end of the absorbent section for securing to each other around a wearer of the garment, the second end of the absorbent section including a fastening device for securing the second end of the absorbent section to the waist belt; and
affixing instructions of application of the garment onto the garment.

2. The method of claim 1, wherein the instructions are directly printed on the garment.

3. The method of claim 1, wherein the instructions are printed on a separate sheet of material and affixed to the garment.

4. The method of claim 3, wherein the instructions are removable.

5. The method of claim 3, wherein the instructions are permanent.

6. The method of claim 1, wherein the instructions are printed on the outside of the garment.

7. The method of claim 1, wherein the instructions are printed on the inside of the garment.

8. The method of claim 7, wherein the instructions are printed in a substance that changes state when wetted by dissolving, smearing or changing color.

9. The method of claim 8, wherein the print acts as a wetness indicator.

10. The method of claim 1, wherein the instructions are printed along a longitudinal extent the garment.

11. The method of claim 1, wherein the instructions are printed along a transversal extent the garment.

12. The method of claim 11, wherein the instructions are printed along a waist edge along the transversal extent of the garment.

13. The method of claim 1, wherein the instructions may be repeated at least two times.

14. The method of claim 13, wherein the instructions may be placed at multiple locations.

15. A garment, comprising:
an absorbent section, the absorbent section having first and second ends;
a waist belt attached to the absorbent section, the waist belt having first and second belt portions extending on each side of the first end of the absorbent section for securing to each other around a wearer of the garment, the second end of the absorbent section including a fastening device for securing the second end of the absorbent section to the waist belt; and
instructions provided on the garment for describing how the garment should be applied.

16. The garment of claim 15, wherein the instructions are directly printed on the garment.

17. The garment of claim 15, wherein the instructions are printed on a separate sheet of material and affixed to the garment.

18. The garment of claim 17, wherein the instructions are removable.

19. The garment of claim 17, wherein the instructions are permanent.

20. The garment of claim 15, wherein the instructions are printed on the outside of the garment.

21. The garment of claim 15, wherein the instructions are printed on the inside of the garment.

22. The garment of claim 21, wherein the instructions are printed in a substance that changes state when wetted by dissolving, smearing or changing color.

23. The garment of claim 22, wherein the print acts as a wetness indicator.

24. The garment of claim 15, wherein the instructions are printed along a longitudinal extent the garment.

25. The garment of claim 15, wherein the instructions are printed along a transversal extent the garment.

26. The garment of claim 25, wherein the instructions are printed along a waist edge along the transversal extent of the garment.

27. The garment of claim 15, wherein the instructions may be repeated at least two times.

28. The garment of claim 27, wherein the instructions may be placed at multiple locations.

29. An absorbent article, comprising:
an absorbent section, the absorbent section having a first and second end;
a fastening device attached to the absorbent section for securing the first end of the absorbent section to the second end of the absorbent section; and
instructions provided on the article for describing how the article should be applied.

30. The absorbent article of claim 29, wherein the instructions are directly printed on the absorbent article.

31. The absorbent article of claim 29, wherein the instructions are printed on a separate sheet of material and affixed to the absorbent article.

32. The absorbent article of claim 31, wherein the instructions are removable.

33. The absorbent article of claim 31, wherein the instructions are permanent.

34. The absorbent article of claim 29, wherein the instructions are printed on the outside of the absorbent article.

35. The absorbent article of claim 29, wherein the instructions are printed on the inside of the absorbent article.

36. The absorbent article of claim 35, wherein the instructions are printed in a substance that changes state when wetted by dissolving, smearing or changing color.

37. The absorbent article of claim 36, wherein the print acts as a wetness indicator.

38. The absorbent article of claim 29, wherein the instructions are printed along a longitudinal extent the absorbent article.

39. The absorbent article of claim 29, wherein the instructions are printed along a transversal extent the absorbent article.

40. The absorbent article of claim 29, wherein the instructions may be repeated at least two times.

41. The absorbent article of claim 40, wherein the instructions may be placed at multiple locations.

42. The method of claim 1, wherein the first end of the absorbent section is located at a rear portion.

43. The method of claim 1, wherein the first end of the absorbent section is located at a front portion.

44. The garment of claim 15, wherein the first end of the absorbent section is located at a rear portion.

45. The garment of claim 15, wherein the first end of the absorbent section is located at a front portion.
